(19) United States
(12) Patent Application Publication
(10) Pub. No.: US 2023/0056630 A1
(43) Pub. Date: Feb. 23, 2023

(54) METHOD OF PRODUCING POLYCAPROLACTONE POWDER BY REPRECIPITATION AND SUBSEQUENT USE OF SAME IN ADDITIVE MANUFACTURING

(71) Applicant: JABIL INC., St. Petersburg, FL (US)

(72) Inventors: Thomas George Gardner, St. Petersburg, FL (US); Victoria Hannah Pyle, St. Petersburg, FL (US); Travis Lee Hislop, St. Petersburg, FL (US)

(21) Appl. No.: 17/820,358

(22) Filed: Aug. 17, 2022

Related U.S. Application Data
(60) Provisional application No. 63/234,812, filed on Aug. 19, 2021.
(60) Provisional application No. 63/265,641, filed on Dec. 17, 2021.

(51) Int. Cl. C08G 63/08

(52) U.S. Cl. CPC C08G 63/08

(57) ABSTRACT

Disclosed is a method of preparing a polycaprolactone powder possessing properties making it well-suited to powder bed fusion 3D printing processes. The polycaprolactone powder disclosed herein has an enthalpy of fusion between 80 J/g and 140 J/g. The polycaprolactone powder described herein has a D₉₀ between 20 microns and 150 microns. The polycaprolactone powder described herein contains a detectable amount of a biocompatible solvent, a bioresorbable solvent, and/or ethyl lactate.

### CROSS REFERENCE TO RELATED PATENT APPLICATIONS

This patent application claims priority to United States Provisional Patent Application No. 63/234,812, filed on Aug. 19, 2021, and U.S. Provisional Patent Application No. 63/265,641, filed on Dec. 17, 2021, which are incorporated by reference herein in their entirety.

### FIELD

The field of the disclosure generally relates to method of producing polycaprolactone in powder form via precipitation from a solution thereof and employing the precipitated polycaprolactone powder in a powder-based additive manufacturing process.

### BACKGROUND

This section provides background information related to this disclosure which is not necessarily prior art.

Various additive manufacturing processes, also known as three-dimensional (3D) printing processes, may be used to form three-dimensional objects by fusing certain materials at particular locations and/or in layers. Material may be joined or solidified under computer control, for example working from a computer-aided design (CAD) model, to create a three-dimensional object, with material being added together, such as liquid molecules or powder grains being fused together, typically layer-by-layer. Various types of additive manufacturing include binder jetting, directed energy deposition, material extrusion, material jetting, powder bed fusion (PBF), sheet lamination, and vat photopolymerization.

Certain additive manufacturing methods may be conducted using thermoplastic polymers; such methods include material extrusion, fused deposition modeling, and PBF. PBF, in general, involves selective fusing of materials in a powder bed. The method may fuse parts of a layer of powder material, move upward in a working area, add another layer of powder material, and repeat the process until an object is built up therefrom. The PBF process may use unfused media to support overhangs and thin walls in the object being produced, which may reduce the need for temporary auxiliary supports in forming the object. PBF 3D print applications may include: SLS (selective laser sintering), MJF (multi jet fusion), HSS (high speed sintering), and electrophotography. In selective heat sintering, a thermal printhead may apply heat to layers of powdered thermoplastic; when a layer is finished, the powder bed moves down, and an automated roller adds a new layer of material which is sintered to form the next cross-section of the object. Selective laser sintering is another PBF process that may use one or more lasers to fuse powdered thermoplastic polymers into the desired three-dimensional object.

Materials for PBF processes may have a uniform shape, size, and composition. The preparation of such powders from thermoplastic polymers on an economical and large scale is not straightforward. What is more, it may be difficult to use amorphous or poorly semi-crystalline polymers, particularly in powder bed fusing processes such as selective laser sintering, because such polymers may not exhibit a sharp melting point. This property may result in dissipation of the applied thermal energy source (e.g., a laser beam) into the regions surrounding where the energy source contacts or strikes the powder bed. This undesired dissipation of thermal energy may result in unstable processing as well as poor feature resolution in the intended three-dimensional object being produced.

Polycaprolactone is known to those skilled in the arts of implantable medical devices and biomaterials as a biocompatible and bioresorbable material that may be used to make medical implants that are not rejected by the human body and which gradually depolymerize and are resorbed by the organism body over time. There is an advantage to being able to produce PCL powder for the purpose of 3D printing implantable medical devices.

It is known to those skilled in the art that PCL powder may be made through grinding or milling processes or a combination of the two. Such methods of preparing PCL powder for use in powder bed fusion (PBF) processes still present several technical issues. In particular, such methods of processing PCL powder into a form suitable for use in certain processes, such as selective laser sintering (SLS), multi jet fusion (MJF), high speed sintering (HSS), and electrophotographic 3D-printing applications, tend to form particles with shapes that deviate far from spheroidal, and also yield an unacceptably wide dispersity of size; therefore, such powders have poor flow characteristics, making them unfavorable for use in PBF processes.

There is a need to provide new and improved methods of making/forming PCL powder.

### SUMMARY OF ILLUSTRATIVE VARIATIONS

A number of variations within the scope of the claims may include processes, compositions, and articles of manufacture that relate to preparation of a PCL powder and its use thereof in additive manufacturing processes, including PBF processes.

At least one variation may include a powder comprising polycaprolactone particles. The powder having greater than 90 volume percent of the particles with a particle size between 20 microns and 150 microns. The powder having a detectable amount of solvent, where the solvent is a biocompatible solvent, a bioresorbable solvent, and/or ethyl lactate. In some variations, greater than 90 volume percent of the polycaprolactone particles have a sphericity greater than 0.75. In another variation, greater than 90 volume percent of the polycaprolactone particles have a sphericity greater than 0.80. In some variations, the volume percent of polycaprolactone particles having a particle size less than 20 microns is zero or undetectable. In some variations, the powder has a peak melting temperature of about 55° C. to about 65° C. and an enthalpy of fusion of about 90 J/g to about 120 J/g.

At least one variation may include a method of preparing PCL powder that may include combining polycaprolactone and dissolving bulk PCL in a polar organic solvent to form a first solution of dissolved polymer at a first temperature; the first temperature may be ambient temperature or greater. The first solution may be then cooled to a second temperature, where the second temperature is lower than the first temperature. A majority portion of the dissolved PCL precipitates as powder from the first solution either en route to, or upon arrival at, the second temperature. The powder is separated from the solution, leaving behind a second, more dilute PCL solution, as well as contaminants from the raw PCL; for example, residual catalyst, initiator, polymerization solvent, monomer, and oligomers. The separated powder may then be washed and dried.

At least one variation may include a method of producing a powder comprising polycaprolactone particles including combining polycaprolactone and a polar organic solvent and dissolving the polycaprolactone in the polar organic solvent along with at least one nucleator. The solution may then be cooled to a lower temperature causing at least a portion of the dissolved polycaprolactone to precipitate in the solution. The precipitated polycaprolactone is separated from the solution, washed, and dried. In some variations, the method includes heating the solution.

In some variations, the polar organic solvent is selected from the group consisting of: ethyl acetate, ethyl lactate, γ-valerolactone, N,N-dimethylformamide (DMF), N-methyl-2-pyrrolidone (NMP), tetrahydrofuran (THF), dichloromethane (DCM), chloroform; acetone, and dimethyl sulfoxide (DMSO). In some variations, the polar organic solvent is ethyl lactate.

In some variations, the method further includes heating the polar organic solvent prior to addition of bulk PCL or after addition of bulk PCL. In some variations the method further includes a second separating step after drying to separate dry polycaprolactone particles having a particle size less than 150 microns from larger dry polycaprolactone particles to form a sized polycaprolactone. In some variations, the method further includes adding a nucleator in powder form to the solution to induce precipitate formation. In some variations, the method further includes adding a secondary solvent to the solution to induce precipitate formation. The secondary solvent being miscible in the polar organic solvent but does not dissolve PCL.

At least one variation may include a method of additive manufacturing including selectively melting or sintering adjacent polycaprolactone particles. Greater than 95 number percent of the polycaprolactone particles have a particle size less than 125 microns, and greater than 90 volume percent of the polycaprolactone particles have a sphericity greater than 0.75. The polycaprolactone particles contain a detecetable amount of ethyl lactate.

At least one variation may include an article that includes polycaprolactone particles. Greater than 90 volume percent of the polycaprolactone particles have a particle size that is between 20 microns and 150 microns. The polycaprolactone particles contain a detectable amount of a solvent comprising at least one of a biocompatible solvent or a bioresorbable solvent.

At least one variation may include a medical product that includes polycaprolactone particles. Greater than 90 volume percent of the polycaprolactone particles have a particle size that is between 20 microns and 150 microns. The polycaprolactone particles contain a detectable amount of a solvent comprising at least one of a biocompatible solvent or a bioresorbable solvent.

Powder compositions for use in PBF processes are provided that include PCL powder prepared by such a method. Objects may be prepared by using such PCL powders in a PBF process to form the object.

The disclosed illustrative of variations of apparatuses, systems, and methods provide PCL powder having suitable properties and characteristics for use in SLS, MJF, HSS, and electrophotography 3D-printing applications. An embodiment of the disclosure may provide a precipitated PCL powder formed through precipitating the polymer from a solvent and then employing the precipitated pulverulent polymer in a powder-based 3D-printing process.

Further areas of applicability will become apparent from the description provided herein. The description and specific examples in this summary are intended for purposes of illustration only and are not intended to limit the scope of the disclosure or claims.

### BRIEF DESCRIPTION OF THE DRAWINGS

The drawings described herein are for illustrative purposes only of selected variations/embodiments and are not intended to limit the scope of the disclosure or claims.

FIG. 1 is a process flow diagram for producing polycaprolactone powder in accordance with an embodiment of the present invention.

FIG. 2 is a graph showing differential scanning calorimetry results of raw PCL used in Example 1 prior to reprecipitation.

FIG. 3 is a graph showing differential scanning calorimetry results of PCL powder reprecipitated from ethyl lactate, in accordance with an embodiment of the present invention.

FIG. 4 is a graph showing particle size volume distribution for PCL powder reprecipitated from ethyl lactate, in accordance with an embodiment of the present invention.

FIG. 5 is a graph showing particle size number distribution for PCL powder reprecipitated from ethyl lactate, in accordance with an embodiment of the present invention.

FIG. 6 is an optical micrograph of PCL powder reprecipitated from ethyl lactate, in accordance with an embodiment of the present invention.

### DETAILED DESCRIPTION OF ILLUSTRATIVE VARIATIONS

The following description is merely illustrative in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is illustrative in nature, and thus, the order of the steps may be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

The term ‘or” as used herein, with respect to a list of two or more items, elements, components, or materials, is not indicative of a complete disjunction such that the listed items, elements, components, or materials are mutually exclusive of each other. For example, “X, Y, or Z” does not mean that each of X, Y, Z are mutually exclusive of each other. Two or more of X, Y, Z could partially or completely overlap each other or that at least one of X, Y, or Z could be included in or be a subgenus of at least one of another of X, Y, or Z. As another example, “cells may be grown in monolayer, three dimensions, or on beads” does not mean that cells grown on beads does not include cells grown in three dimensions. As a further example, “at least one of a biocompatible solvent; a bioresorbable solvent; or ethyl lactate” does not mean that ethyl lactate nor a solvent including ethyl lactate is neither a biocompatible solvent nor a bioresorbable solvent; nor does it mean that a biocompatible solvent or a bioresorbable solvent cannot be or include ethyl lactate.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9,1-8,1-3,1-2,2-10,2-8,2-3,3-10,3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

The particle size of the PCL polymer may affect its use in additive manufacturing processes. As used herein, D₅₀ (as known as “volume median diameter” or “average particle diameter by volume”) refers to the particle diameter of the powder where 50 vol. % of the particles in the total distribution of the referenced sample have the noted particle diameter or smaller. Similarly, D₁₀ refers to the particle diameter of the powder where 10 vol. % of the particles in the total distribution of the referenced sample have the noted particle diameter or smaller; and D₉₀ refers to the particle diameter of the powder where 90 vol. % of the particles in the total distribution of the referenced sample have the noted particle diameter or smaller. Particle sizes may be measured by any suitable methods known in the art to measure particle size by diameter. The semi-crystalline polymer powder provided herein may have a D90 particle size of less than 150 μm.

As used herein, “layer” is a term of convenience that includes any shape, regular or irregular, having at least a predetermined thickness. In certain embodiments, the size and configuration two dimensions are predetermined, and in certain embodiments, the size and shape of all three-dimensions of the layer are predetermined. The thickness of each layer may vary widely depending on the additive manufacturing method. In certain embodiments the thickness of each layer as formed may differ from a previous or subsequent layer. In certain embodiments, the thickness of each layer may be the same. In certain embodiments the thickness of each layer as formed may be from 0.5 millimeters (mm) to 5 mm.

Certain variations may include forming a plurality of layers in a preset pattern by an additive manufacturing process. In a number of variations, the additive manufacturing may produce two or more layers, or 20 or more layers. The maximum number of layers may vary greatly, determined, for example, by considerations such as the size of the object being manufactured, the technique used, the capacities and capabilities of the equipment used, and the level of detail desired in the final object. For example, 5 to 100,000 layers may be formed, or 20 to 50,000 layers may be formed, or 50 to 50,000 layers may be formed.

The term “powder bed fusing” or “powder bed fusion” is used herein to mean processes wherein the polymer is selectively sintered or melted and fused, layer-by-layer to provide a 3-D object. Sintering may result in objects having a density of less than about 90% of the density of the solid powder composition, whereas melting may provide objects having a density of 90%-100% of the solid powder composition. Use of semi-crystalline polymer as provided herein may facilitate melting such that resulting densities may approach densities achieved by injection molding methods.

Powder bed fusing or powder bed fusion further includes all laser sintering and all selective laser sintering processes as well as other powder bed fusing technologies as defined by ASTM F2792-12a. For example, sintering of the powder composition may be accomplished via application of electromagnetic radiation other than that produced by a laser, with the selectivity of the sintering achieved, for example, through selective application of inhibitors, absorbers, susceptors, or the electromagnetic radiation (e.g., through use of masks or directed laser beams). Any other suitable source of electromagnetic radiation may be used, including, for example, infrared radiation sources, microwave generators, lasers, radiative heaters, lamps, or a combination thereof. In certain embodiments, selective mask sintering (“SMS”) techniques may be used to produce three-dimensional objects. For further discussion of SMS processes, see for example U.S. Pat. No. 6,531,086, incorporated herein by reference in its entirety, which describes an SMS machine in which a shielding mask is used to selectively block infrared radiation, resulting in the selective irradiation of a portion of a powder layer. If using an SMS process to produce objects from powder compositions of the present technology, it may be desirable to include one or more materials in the powder composition that enhance the infrared absorption properties of the powder composition. For example, the powder composition may include one or more heat absorbers (e.g., glass fibers or glass microbeads) or dark-colored materials (e.g., carbon black, carbon nanotubes, or carbon fibers).

Also included herein are all three-dimensional objects made by powder bed fusing compositions including the semi-crystalline polymer powder described herein. After a layer-by-layer manufacture of an object, the object may exhibit excellent resolution, durability, and strength. Such objects may include various articles of manufacture that have a wide variety of uses, including uses as prototypes, as end products, as well as molds for end products.

An object may be formed from a preset pattern, which may be determined from a three-dimensional digital representation of the desired object as is known in the art and as described herein. Material may be joined or solidified under computer control, for example, working from a computer-aided design (CAD) model, to create the three-dimensional object.

In particular, powder bed fused (e.g., laser sintered) objects may be produced from compositions including PCL powder using any suitable powder bed fusing processes including laser sintering processes. These objects may include a plurality of overlying and adherent sintered layers that include a polymeric matrix which, in some embodiments, may have reinforcement particles dispersed throughout the polymeric matrix. Laser sintering processes are known, and are based on the selective sintering of polymer particles, where layers of polymer particles are briefly exposed to laser energy and the polymer particles exposed to the laser energy are thus bonded to one another. Successive sintering of layers of polymer particles produces three-dimensional objects. Details concerning the selective laser sintering process are found, by way of example, in the specifications of U.S. Pat. No. 6,136,948 and WO 96/06881, the entire contents of each of which are incorporated herein by reference. However, the semi-crystalline polymer powder described herein may also be used in other rapid prototyping or rapid manufacturing processing of the prior art, in particular in those described above. For example, the semi-crystalline polymer powder may in particular be used for producing moldings from powders via the SLS (selective laser sintering) process, as described in U.S. Pat. No. 6,136,948 or WO 96/06881, via the SIB process (selective inhibition of bonding of powder), as described in WO 01/38061, via 3D printing, as described in EP 0 431 924, or via a microwave process, as described in DE 103 11 438, the entire contents of each of which are incorporated herein by reference.

The fused layers of powder bed fused objects may be of any thickness suitable for selective laser sintered processing. The individual layers may be each, on average, at least 50 μm thick, at least 80 μm thick, or at least 100 μm thick. In a number of variations, the plurality of sintered layers are each, on average, less than 500 μm thick, less than 300 μm thick, or less than 200 μm thick. Thus, the individual layers for some embodiments may be 50 to 500 μm, 80 to 300 μm, or 100 to 200 μm thick. Three-dimensional objects produced from powder compositions of the present technology using a layer-by-layer powder bed fusing processes other than selective laser sintering may have layer thicknesses that are the same or different from those described above.

A number of variations may provide ways to make and use PCL powder having suitable characteristics for use in selective laser sintering (SLS), multi jet fusion (MJF), high speed sintering (HSS), and electrophotographic (EPG) 3D-printing. At least one variation may provide a precipitated PCL powder formed through precipitation of the polymer from a saturated solution of PCL in a polar organic solvent, allowing the polymer to form crystallites, and then employing the precipitated polymer powder in a PBF 3D-printing process. A number of variations of PCL powder may exhibit optimized characteristics for PBF processes, including optimized particle size and dispersity thereof, shape, and crystallinity, while at the same time using a dispersant-free single-solvent process in the manufacture thereof.

Methods of preparing PCL powder may include dissolving bulk PCL in ethyl lactate to form a solution at elevated temperature; cooling the solution to room temperature to form a PCL powder as a precipitate having a D₉₀ value of less than 150 micrometers (microns, or μm); a D₅₀ value of less than or equal to 100 μm, or a D₅₀ value of between 0 to 100 μm. The methods may also yield a product where the particles may exhibit a certain size (about 30 μm to about 40 μm in average diameter), low dispersity, spheroidal shape, and crystalline character suitable for the above-mentioned printing processes in comparison to the results of aforementioned processes. The act of reprecipitation also serves to purify the PCL.

Powder compositions for use in PBF processes are provided that include PCL powder prepared by such a method. Objects may be prepared by using such PCL powders in a PBF process to form the object.

In certain embodiments, a method of preparing PCL powder is provided that includes dissolving bulk PCL in a polar solvent such as an ester; for example, ethyl lactate, to form a first solution of dissolved polymer at a first temperature. The first solution is then cooled to a second temperature, where the second temperature is lower than the first temperature. A portion of the dissolved PCL precipitates as powder from the first solution either en route to, or upon arrival at, the second temperature, leaving behind a second, more dilute PCL solution. The precipitated PCL powder may be separated from a remainder of the second solution, effected for example by gravity filtration, vacuum filtration, or centrifugation. The separated PCL powder may also be washed with water or an organic solvent, provided the wash solvent is miscible with the solvent used for reprecipitation, and that the wash solvent does not dissolve the polymer powder to a deleterious extent (e.g., unacceptably excessive loss of material and/or unacceptably excessive reduction of particle size), and may not a solvent for the polymer powder product at all. The separated PCL powder may also be dried, subsequent to any washing procedure, if applied. In certain embodiments, the polar solvent may include ethyl lactate. In other embodiments, the polar solvent may consist essentially of ethyl lactate. And in still further embodiments, the polar solvent may consist of ethyl lactate.

Various solvent temperatures may be employed in methods of preparing PCL powder by reprecipitation. The dissolving step may include heating PCL in a polar solvent to form the first solution of dissolved PCL at the first temperature, where the first temperature is greater than room temperature. The cooling step may include cooling the first solution to the second temperature, where the second temperature is below the precipitation temperature of the polymer solution, and may be at ambient temperature (“room temperature”) or lower. Ambient (“room”) temperature is understood to be about 20-25° C. (68-77° F.).

Various embodiments of PCL may exhibit the following physical characteristics. The PCL powder may have a D₉₀ particle size of less than about 150 μm. In certain embodiments, the PCL powder may have a D₅₀ of less than about 100 μm. The PCL powder may also have a D₅₀ value from about 1 micrometer to about 100 μm. Particular embodiments include where the PCL powder has a D₅₀ value from about 30 μm to about 40 μm. The PCL powder may be in the form of spheroidal particles.

Melting point and enthalpy of fusion for the polymer powder may be determined using differential scanning calorimetry (DSC); for example, a TA Instruments Discovery Series DSC 250 scanning at 20° C./min.

Percent crystallinity of a polymer may be determined by the ratio of the enthalpy of fusion, as measured by DSC, to the enthalpy of fusion of a theoretical 100% crystalline polymer, which for PCL is reported as having a value of 139.5 J/g (Gupta and Geeta, J. Appl. Polym. Sci. 2012, 123(4), 1944-1950). Percent crystallinity may also be determined directly by powder x-ray crystallography and correlated to enthalpy of fusion in a directly linear relationship.

Powder flow for the polymer powder may be measured using Method A of ASTM D₁₈₉₅ and was determined using a cone with a 10 mm nozzle diameter.

In some embodiments, the particle size of the polymer powder is determined by laser diffraction as is known in the art. For example, particle size may be determined using a laser diffractometer such as the Microtrac S3500.

In certain embodiments, powder compositions for use in a PBF 3D printing process are provided, where such powder compositions include PCL powder prepared according to the methods provided herein. For example, a powder composition for use in a PBF process may include PCL powder having a D₉₀ particle size of less than about 150 μm, and a D₅₀ value from about 30 μm to about 40 μm. Such powder compositions may include mixtures of PCL powders having different physical characteristics as well as additives and other components as described herein.

In certain embodiments, reprecipitated PCL powder prepared by methods disclosed herein is used in a PBF 3D printing process to form an object. Certain methods of preparing an object include providing PCL powder having a D₉₀ particle size of less than about 150 μm, a D₅₀ value from about 30 μm to about 40 μm. The PCL powder is then used in a PBF process to form the object.

In certain embodiments, one or more objects prepared by an additive manufacturing process are provided. Such methods may include providing PCL powder prepared according to one or more of the methods described herein. The PCL powder is then used in a PBF process to form the one or more objects.

Certain embodiments may include methods for powder bed fusing that use a powder composition including PCL powder to form a three-dimensional object. Due to the good flowability of reprecipitated PCL powder, a smooth and dense powder bed may be formed allowing for optimum precision and density of the sintered object.

In certain embodiments, the method of preparing PCL powder comprises dissolving bulk PCL in a polar solvent such as ethyl lactate at a temperature above room temperature. Ambient (“room”) temperature is understood to be about 20-25° C. (68-77° F.); as such, the PCL may be dissolved in ethyl lactate above ambient temperature. The PCL is soluble in the ethyl lactate solvent and thus a PCL solution is formed. In general, the solution may be prepared at a temperature above room temperature so that the amount of dissolved PCL is greater than what the solvent is capable of keeping in solution at ambient temperature. Mixing of PCL into ethyl lactate solvent may be carried out in-line or batch. The process may readily be carried out at manufacturing scale. Upon cooling to room temperature (e.g., about 20° C.), the dissolved PCL begins to crystallize and precipitate out of the ethyl lactate solvent resulting in the precipitation of a PCL precipitate.

Following precipitation, the ethyl lactate solvent is removed, for example by filtration or centrifugation. The PCL powder may then be washed with a solvent that is miscible with the reprecipitation solvent and reasonably volatile, for example, water, filtered to remove the wash solvent, and dried with or without application of heat, and with or without application of vacuum. It is further advantageous to use a wash solvent in which PCL is minimally soluble or insoluble.

As provided herein, PCL is dissolved in a polar organic solvent. For example, PCL may be dissolved in the solvent under conditions that result in a saturated solution of PCL, where changing conditions (e.g., lowering the temperature of the solution) result in precipitation of PCL powder therefrom. In certain embodiments, the solvent may include ethyl lactate as well as one or more other esters or one or more other polar organic solvents. In certain embodiments, the solvent may consist essentially of ethyl lactate, where no other components are present that materially affect the crystallization of PCL. In certain embodiments, the solvent may be substantially 100% ethyl lactate. It is further noted that upon precipitating PCL powder from a solution of PCL in ethyl lactate, a portion of the dissolved PCL may remain in solution. In certain embodiments, the addition of a secondary solvent which is miscible with the reprecipitation solvent but does not support dissolution of the PCL may be added to the PCL/solvent solution to induce precipitation. In certain embodiments, the use of a nucleating agent in powder form may be used to induce precipitation, and may help to control particle size and dispersity of particle size, and may help to improve the overall spheroidal shape of the powder particles. Separation of the precipitated PCL powder from the remainder of the solution therefore leaves a solution of ethyl lactate with a portion of dissolved PCL.

Ethyl lactate is a useful solvent for the process in that it dissolves PCL well; is shown herein to produce powder with characteristics well-suited to PBF 3D printing processes; has a boiling point well-separated from ambient temperature, allowing for a broad cooling range during precipitation; is miscible with commonly available and effective wash solvents (e.g., water or low molecular weight alcohols); has been shown to be relatively non-toxic in mammals (as exhibited in its use as a food additive); and may be broken down in the body to form ethanol and lactic acid.

In certain embodiments, the precipitated PCL powder has a D₈₅ particle size of less than 150 μm; specifically, a D₉₀ particle size of less than 150 μm. Certain embodiments include where the PCL powder has a D₉₀ particle size of less than 150 μm. A PCL powder in which 100% of the particles have a size of less than 150 μm may also be produced by this method. The PCL powder may also have a D₅₀ value of less than or equal to 100 μm. Specifically, the PCL powder may have a D₅₀ value of 10 μm to 100 μm. The average particle diameter of the PCL powder may also be less than or equal to 100 μm or include a D₅₀ value of between 0 to 100 μm.

In certain embodiments, a method of preparing an article comprises providing a powder composition comprising PCL powder, and using a powder bed fusing process with the powder composition to form a three-dimensional object. At least one PCL powder may have a D₅₀ particle size of less than 150 μm in diameter and is made by above-described methods. Embodiments include where the PCL powder has a D₉₀ particle size of less than 150 μm, a D₅₀ value of less than or equal to 100 μm, or a D₅₀ value of between 0 to 100 μm.

The PCL powder may be used as the sole component in the powder composition and applied directly in a powder bed fusing step. Alternatively, the PCL powder may first be mixed with other polymer powders, for example, another crystalline polymer or an amorphous polymer, or a combination of a semi-crystalline polymer and an amorphous polymer. The powder composition used in the powder bed fusing may include between 50 wt % to 100 wt % of the PCL powder, based on the total weight of all polymeric materials in the powder composition.

The PCL powder may also be combined with one or more additives/components to make a powder useful for powder bed fusing methods. Such optional components may be present in a sufficient amount to perform a particular function without adversely affecting the powder composition performance in powder bed fusing or the object prepared therefrom. Optional components may have a D₅₀ value which falls within the range of the average particle diameters of the PCL powder or an optional flow agent. If necessary, each optional component may be milled to a desired particle size and/or particle size distribution, which may be substantially similar to the PCL powder. Optional components may be particulate materials and include organic and inorganic materials such as fillers, flow agents, and coloring agents. Still other additional optional components may also include, for example, toners, extenders, fillers, colorants (e.g., pigments and dyes), lubricants, anticorrosion agents, thixotropic agents, dispersing agents, antioxidants, adhesion promoters, light stabilizers, organic solvents, surfactants, flame retardants, anti-static agents, plasticizers a combination comprising at least one of the foregoing. Yet another optional component also may be a second polymer that modifies the properties of the PCL powder. In certain embodiments, each optional component, if present at all, may be present in the powder composition in an amount of 0.01 wt % to 30 wt %, based on the total weight of the powder composition. The total amount of all optional components in the powder composition may range from 0 up to 30 wt % based on the total weight of the powder composition. Such an additive may also enhance the conversion of IR laser energy into thermal energy in the powder bed.

It is not necessary for each optional component to melt during the powder bed fusing process; e.g., a laser sintering process. However, each optional component may be selected to be homogeneously compatible with the PCL polymer in order to form a strong and durable object. The optional component, for example, may be a reinforcing agent that imparts additional strength to the formed object. Examples of the reinforcing agents include one or more types of glass fibers, carbon fibers, talc, clay, wollastonite, glass beads, and combinations thereof. Such an additive may also enhance the conversion of IR laser energy into thermal energy in the powder bed.

The powder composition may optionally contain a flow agent. In particular, the powder composition may include a particulate flow agent in an amount of 0.01 wt % to 5 wt %, specifically, 0.05 wt % to 1 wt %, based on the total weight of the powder composition. In certain embodiments, the powder composition comprises the particulate flow agent in an amount of 0.1 wt % to 0.25 wt %, based on the total weight of the powder composition. The flow agent included in the powder composition may be a particulate inorganic material having a median particle size of 10 μm or less, and may be chosen from a group consisting of hydrated silica, amorphous alumina, glassy silica, glassy phosphate, glassy borate, glassy oxide, titania, talc, mica, fumed silica, kaolin, attapulgite, calcium silicate, alumina, magnesium silicate, and combinations thereof. The flow agent may be present in an amount sufficient to allow the semi-crystalline polymer powder to flow and level on the build surface of the powder bed fusing apparatus (e.g., a laser sintering device). Such an additive may also enhance the conversion of IR laser energy into thermal energy in the powder bed.

The powder composition may optionally contain an IR-absorbing agent to facilitate the conversion of laser energy into thermal energy in the SLS process. The IR-absorbing agent may be one or more of a variety of inorganic or organic substances, such as metal oxides (e.g., titania, silica, glass, tungsten(VI) oxide), metal nanoparticles (e.g., gold nanorods), or organic compounds that absorb strongly at the wavelength of the IR laser (typically 10.6 μm equivalent to 943 cm⁻¹).

Another optional component is a coloring agent, for example a pigment or a dye, like carbon black, to impart a desired color to the object. The coloring agent is not limited, as long as the coloring agent does not adversely affect the composition or an object prepared therefrom, and where the coloring agent is sufficiently stable to retain its color under conditions of the powder bed fusing process and exposure to heat and/or electromagnetic radiation; e.g., a laser used in a sintering process. Such an additive may also enhance the conversion of IR laser energy into thermal energy in the powder bed.

Still further additives include, for example, toners, extenders, fillers, lubricants, anticorrosion agents, thixotropic agents, dispersing agents, antioxidants, adhesion promoters, light stabilizers, organic solvents, surfactants, flame retardants, anti-static agents, plasticizers, and combinations of such. Such an additive may also enhance the conversion of IR laser energy into thermal energy in the powder bed.

Still another optional component also may be a second polymer that modifies the properties of the PCL powder.

The powder composition is a fusible powder composition and may be used in a powder bed fusing process such as selective laser sintering. An example of a selective laser sintering system for fabricating a part from a fusible powder composition, and in particular for fabricating the part from the fusible PCL powder disclosed herein, may be described as follows. One thin layer of powder composition comprising the PCL powder is spread over the sintering chamber. The laser beam traces the computer-controlled pattern, corresponding to the cross-section slice of the CAD model, to melt the powder selectively which has been preheated to slightly below its melting temperature. After one layer of powder is sintered, the powder bed piston is lowered with a predetermined increment (typically 100 μm), and another layer of powder is spread over the previous sintered layer by a roller. The process then repeats as the laser melts and fuses each successive layer to the previous layer until the entire object is completed. Three-dimensional objects comprising a plurality of fused layers may thus be made using the PCL powder described herein.

One or more variation may be constructed and arranged to provide one or more advantages, which may include, but not limited to, the use of a single solvent in preparing the PCL powder, which facilitates solvent recovery and reuse thereof. A number of variations, the PCL powder produced by at least one of the disclosed methods provides improved PBF performance. Additive manufacturing processes that employ fusion of a powder bed, including selective laser sintering (SLS), multi jet fusion (MJF), high speed sintering (HSS), and electrophotographic 3D-printing, may therefore benefit by forming and using PCL powder produced as described herein. In particular, the 3D printing of implantable, bioresorbable medical devices would benefit from the PCL powder material described herein.

In a number of variations, the reprecipitation process may serve to purify the PCL material, removing residual catalyst, initiator, monomer, and other contaminants. By dissolving the PCL, contaminants interstitially trapped in the solid are released into the resulting PCL solution. When the PCL reprecipitates, the quantity of contaminants that become reintercalated into the solid is significantly less, due both to a lower probability of entrapment, as well as the nature of the formation of crystallites to exclude contaminants. The reprecipitation process may be repeated with fresh, uncontaminated solvent to further reduce the level of contamination. A common contaminant to be removed from PCL is the tin compounds residual from the common use of a tin catalyst in the process of polymerizing ε-caprolactone.

A number of variations may include a method of producing powder suitable for additive manufacturing, the method comprising: combining a polymeric material suitable and a solvent; dissolving the polymeric material suitable for additive manufacturing into the solvent to form a solution; cooling the solution to a temperature that causes at least a portion of the dissolved polymeric material suitable for additive manufacturing to precipitate from the solution; separating precipitated polymeric material from the solution;

washing the separated, precipitated polymeric material to form a washed polymeric material; and drying the washed polymeric material to form a dry polymeric material suitable for additive manufacturing.

### EXAMPLE 1

To a 20-L glass reactor containing ethyl lactate (7.0 L) at 120° C. was added polycaprolactone (1.0 kg) and the mixture was allowed to stir at 500-1000 rpm until the polymer was fully dissolved. This step may be performed at ambient (atmospheric) pressure under a blanket of air or an inert gas, e.g., nitrogen or argon. Heating was discontinued to allow the solution to cool to room temperature, during which time precipitation commenced. The slurry was allowed to stir for 16 hours at room temperature before filtering through a 25 μm filter paper in a vacuum filtration apparatus. The recovered wet powder was slurried into water (9.5 L), stirred for one hour at 600-1000 rpm, collected by vacuum filtration as previously described, and allowed to air dry in an evaporation dish at a powder depth on one inch for 72 hours.

Density and Flowability. Average bulk density of the PCL powder was 0.2395 g/cm³. Average tap density of the PCL powder after 2000 taps was 0.2928 g/cm³. These values indicate a Hausner ratio of 1.2228 and a Carr index of 18.219, suggesting the powder has flowability sufficient for good performance in an SLS printer. Flowability was determined using a cone with a 10 mm nozzle diameter, and has an average value of 23.4110 g/sec. The angle of repose was measured to be 35.390°.

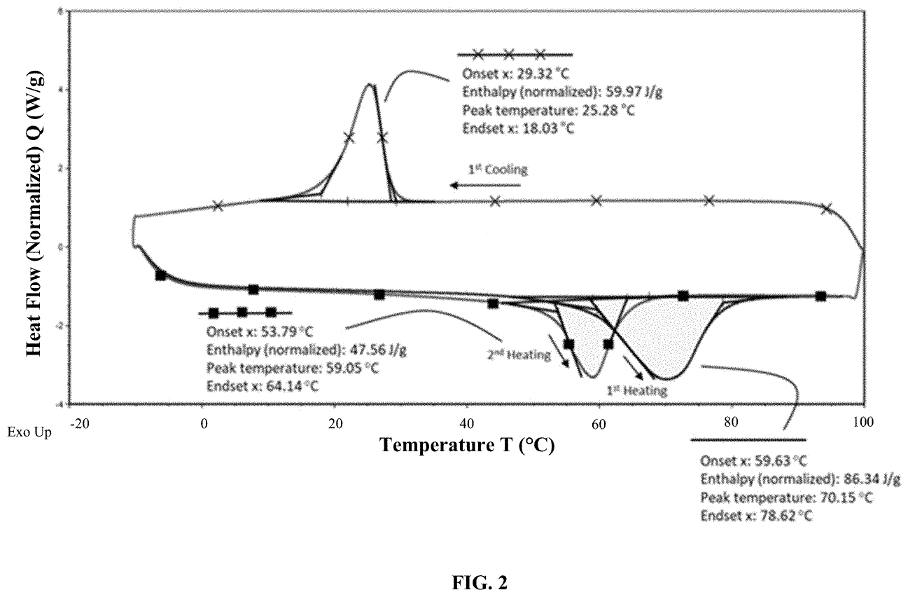

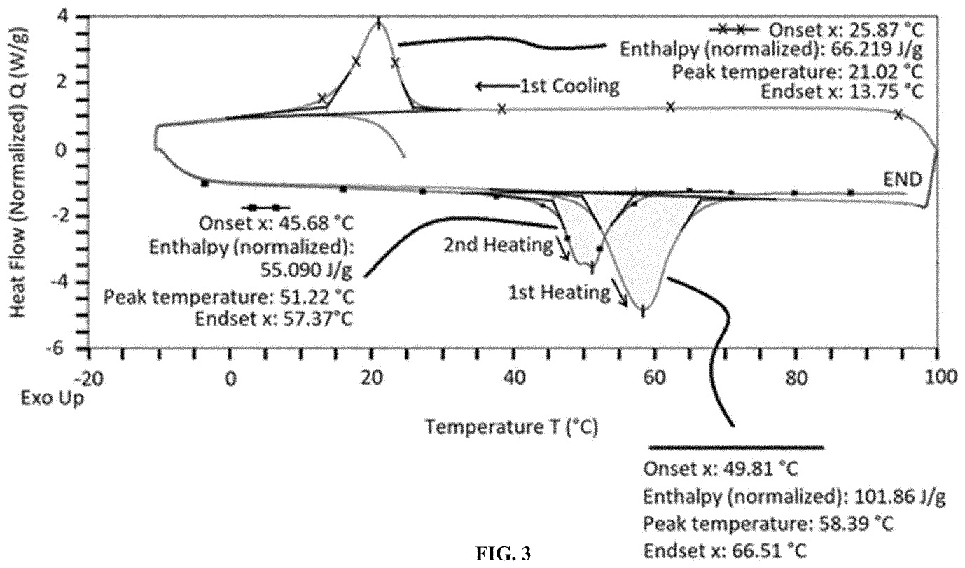

Differential scanning calorimetry (DSC) and Crystallinity. DSC traces for raw PCL and reprecipitated PCL are shown in FIGS. 2 and 3, respectively. The raw PCL has a melt onset temperature of 59.63° C., a peak melt temperature of 70.15° C., and an enthalpy of fusion of 86.34 J/g, which correlates to 61.9% crystallinity. The reprecipitated PCL has a narrower melt curve, with a melt onset temperature of 49.81° C., a peak melt temperature of 58.39° C., and an enthalpy of fusion of 101.86 J/g, which correlates to 73.0% crystallinity.

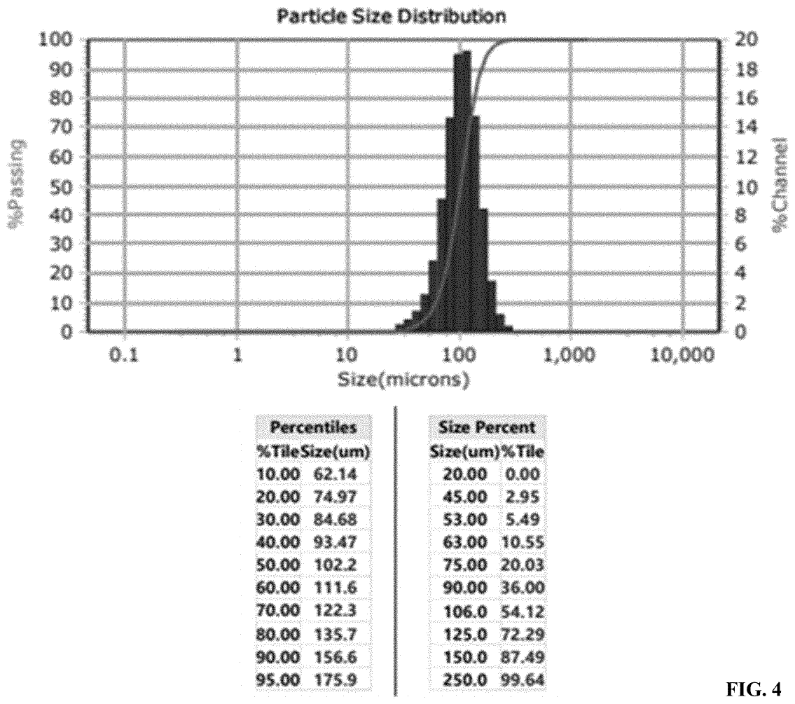

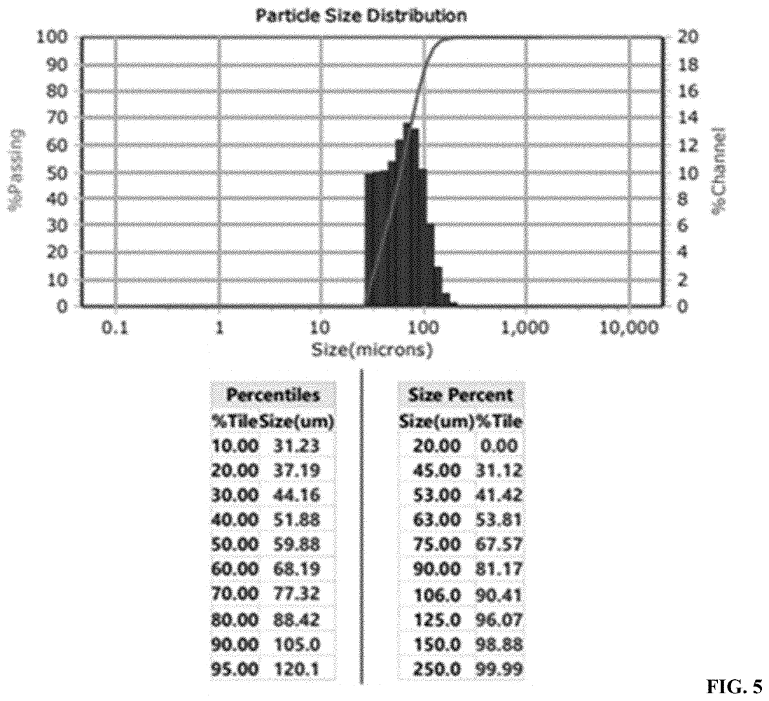

Particle size, shape and distribution (PSSD). PSSD was determined in water, and the particle size volume distribution and particle size number distributions are shown in FIGS. 4 and 5, respectively. Sphericity data shows 90.54% of the sample has sphericity >0.75, and 80.64% of the sample has sphericity >0.80.

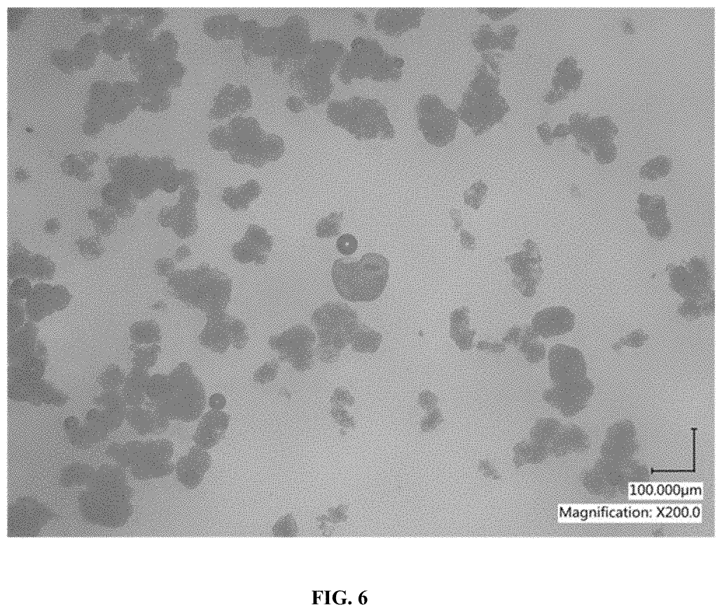

Optical micrography. A representative micrograph image of the powder is shown in FIG. 6.

### Example Process 1

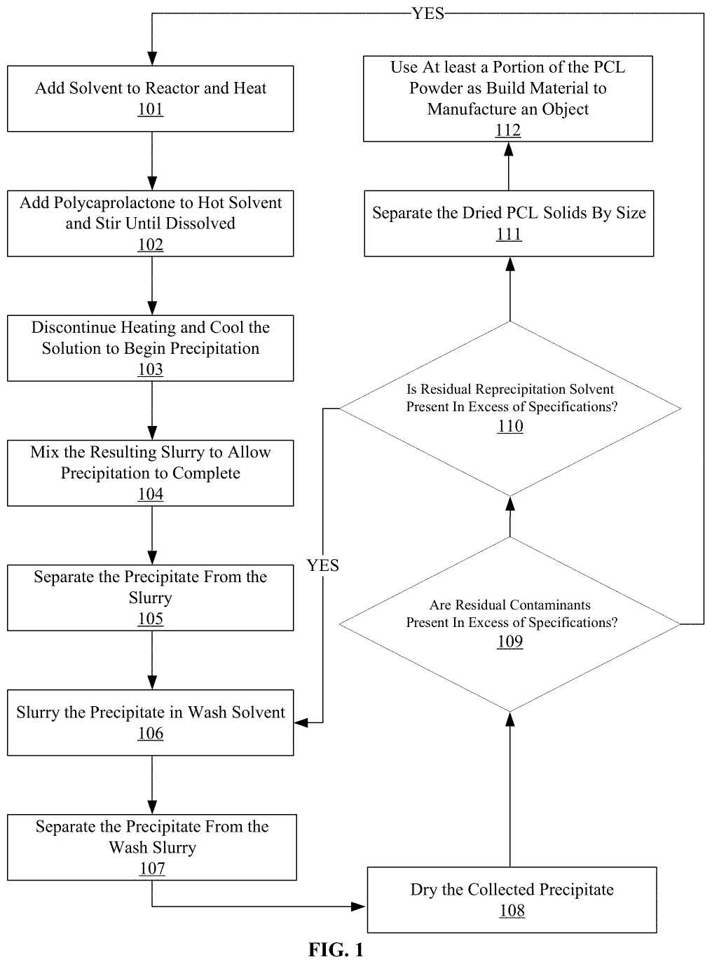

FIG. 1 illustrates a method of producing polycaprolactone powder, according to at least one variation.

At Step 101, the solvent is heated to the desired operating temperature. The temperature of the solvent may be controlled to a setpoint temperature. The temperature of the solvent may be equal to the setpoint temperature. The solvent may be selected from a group including: acyclic and cyclic esters (e.g., ethyl acetate, ethyl lactate, or γ-valerolactone); acyclic and cyclic secondary amides (e.g., N,N-dimethylformamide (DMF) or N-methyl-2-pyrrolidone (NMP)); cyclic ethers (e.g., tetrahydrofuran (THF)); halogenated hydrocarbons (e.g., dichloromethane (DCM) or chloroform); acyclic and cyclic ketones (e.g., acetone); and dimethyl sulfoxide (DMSO). In at least one variation, the solvent is ethyl lactate, and may be used in either an enantiopure or racemic form or a mixture thereof. The solvent may have a temperature that is also above the melting point of PCL. A single solvent may be used; alternatively, a mixture of solvents may be used. The operating temperature may be any temperature between the freezing point and the boiling point of the solvent; in addition, the operating temperature must be higher than the final temperature after the cooling step 103. For example, if ethyl lactate is the selected solvent, the operating temperature may be between 30° C. and 150° C. (including the end points of the range), between 70° C. and 130° C. (including the end points of the range), or between 80° C. and 120° C. (including the end points of the range). The boiling point of an ethyl lactate solvent is about 154° C.

At step 102, PCL is added to the heated solvent and allowed to dissolve completely as judged by visual observation. The temperature of the solvent/PCL combination may be controlled to a setpoint temperature. PCL pieces of any size may be used. PCL may be heated before being added to the solvent to prevent the solvent temperature from decreasing upon PCL addition. In at least one variation, PCL may be heated above its melting point and then added to the solvent. In certain embodiments, the PCL may be added prior to the heating stage of step 101. The PCL/solvent combination may be mixed, for example by stirring. A stir rate of 200 to 1000 revolutions per minute may be used. In at least one variation, a stir rate of 500 to 800 rpm may be used. The concentration of PCL in the solvent may range from 1% w/v to 20% w/v where the concentration of PCL is calculated by dividing the mass of PCL (in kilograms, kg) by the volume of the solvent (in liters, L). In some variations, PCL concentration may be (a) 13% w/v to 15% w/v or (b) 8% w/v to 10% w/v. Step 102 may proceed until all of the PCL is dissolved based on visual inspection. When all the PCL is dissolved, the solution may appear completely transparent and there may be minimal or no visible solids present.

At step 103, the temperature of the PCL solution may be reduced. A cooling step may lower the temperature through and below the saturation point of the solution which may cause dissolved PCL to precipitate out of solution. In certain embodiments, the temperature of the PCL/solvent solution may be reduced to room temperature. In certain embodiments, a nucleator in powder form may be added to induce precipitate formation, as well as may improve particle size and size dispersity, and may also improve particle spheroidal shape characteristics. In certain embodiments, a secondary solvent may be added to the first solvent, and is miscible with the first solvent, but does not support the dissolution of PCL, for the purpose of inducing precipitation of the PCL.

At step 104, the slurry obtained in the previous step is allowed to stir in order to effect further precipitation. The stir rate may be between 500 and 1000 rpm. The length of time for stirring may be at least one hour, and may be between 16 and 24 hours.

At step 105, the precipitated PCL powder may be separated from the supernatant solution. Separation may be accomplished, for example, by vacuum filtration, or by other separation techniques such as screening, centrifugation, cyclone separators, air classification, drying, etc. If a filter medium is used, the porosity of the medium should be of sufficiently small size to minimize loss of particles of the desired sizes; for example, to capture particles suitable for PBF, a filter of porosity between 5 μm and 40 μm may be used, with a porosity of between 20 μm and 30 μm. The filter medium may consist of, for example, paper, fritted glass, polymer mesh, metal mesh, or membrane.

At step 106, the solvent-wet PCL powder is washed. The wash solvent should be miscible with the reprecipitations solvent. The wash solvent should further be a poor or non-solvent for PCL so as to minimize loss of PCL or reduction of particle size as a result of the wash process. The wash solvent should further be reasonably volatile and easily removed in the drying step 108, such that the solvent may be removed easily and in a timely manner by any drying method known in the art; e.g., air drying, convection drying, or drying under vacuum, with or without the application of heat. As an example, a wash solvent to remove ethyl lactate may consist of water or a low molecular weight alcohol such as methanol, ethanol, or isopropanol, or a mixture thereof. The wash solvent may be combined with PCL and the combination may be mixed. Alternatively, wash solvent may be applied, for example by spraying, over PCL powder that is positioned on top of a mesh or screen to remove the reprecipitation solvent and wash it from the PCL. Other liquid displacement or extraction methods may also be used. The wash process may be performed once or repeated as needed.

At step 107, the precipitated PCL powder may be separated from the wash solution. The method of separation may be any of those described for step 105.

At step 108, the PCL may be dried. PCL may be dried by heating to a temperature ranging from ambient to 50° C. PCL may be stationary and allowed to air dry by evaporation without application of heat, convected gases, or vacuum. PCL may be stationary as hot air (or other gas, such as nitrogen) at a temperature less than 50° C. passes over it to carry wash solvent vapor away. Alternatively, PCL may be tumbled or otherwise moved to improve mass transfer of wash solvent from the PCL to the surrounding environment during the drying step. A vacuum system may be used to decrease the pressure that the PCL is exposed to during the drying step to reduce the energy required for drying and/or to achieve more complete drying.

At step 109, an optional quality check may be performed on the dried PCL solid to determine the presence of contaminants; most notably, inorganic residues, and in particular, tin compounds may be residual in the PCL from the use of tin-based catalysts in various processes for polymerizing ε-caprolactone. If such contaminants are detected by any of various analytical methods known to those skilled in the art, step 109A allows for the provision that the powder may be reprecipitated by returning to step 101 and repeating the process form that point using fresh reprecipitation solvent.

At step 110, an optional quality check may be performed on the dried PCL solid to determine the presence of residual reprecipitation solvent, for example, ethyl lactate. If residual solvent is detected by any of various analytical methods known to those skilled in the art, step 110A allows for the provision that the powder may be washed again by returning to step 106 and repeating the process from that point.

At step 111, dried PCL particles may be separated by size. Size separation may separate/isolate PCL particles that have a particle diameter within the range of 30 to 150 μm, 20 to 150 μm, or 1 to 150 μm. Size separation may separate PCL particles that have a particle diameter within a range that is desirable for a particular end use such as SLS printing. Size separation may be accomplished by screening, cyclone separation, air classifier, etc.

Finally, at step 112, the PCL or a sized fraction of the PCL may be used as a build material to manufacture an article. For example, a sized fraction of PCL may be used as a build material in an SLS printer to produce a 3D printed object. In at least one variation, the size separation step is excluded and the powder is used in an end-use application (for example, SLS printing).

What is claimed is:

1. A powder comprising polycaprolactone particles, wherein greater than 90 volume percent of the polycaprolactone particles have a particle size that is between 20 microns and 150 microns and wherein the polycaprolactone particles contain a detectable amount of a solvent comprising at least one of a biocompatible solvent or a bioresorbable solvent.

2. The powder of claim 1, wherein the solvent comprises ethyl lactate.

3. The powder of claim 1, wherein greater than 90 volume percent of the polycaprolactone particles have a sphericity that is greater than 0.75

4. The powder of claim 1, wherein greater than 80 volume percent of the polycaprolactone particles have a sphericity that is greater than 0.80.

5. The powder of claim 1, wherein the volume percent of polycaprolactone particles having a particle size less than 20 microns is zero or undetectable.

6. The powder of claim 1, wherein the powder has an enthalpy of fusion of about 80 J/g to about 140 J/g.

7. A powder comprising polycaprolactone particles and a detectable amount of ethyl lactate, wherein the powder has a peak melting temperature of about 55° C. to about 65° C. and an enthalpy of fusion of about 90 J/g to about 120 J/g.

8. The powder of claim 7, wherein the powder has a recrystallization peak of about 15° C. to about 35° C.

9. The powder of claim 7, wherein the powder has an onset of degradation temperature of about 250° C. to about 425° C.

10. The powder of claim 7, wherein greater than 96 number percent of the polycaprolactone particles have a particle size that is less than 125 microns.

11. The powder of claim 7, wherein greater than 90 volume percent of the polycaprolactone particles have a sphericity that is greater than 0.75.

12. A powder comprising polycaprolactone particles having a detectable amount of ethyl lactate, wherein greater than 95 number percent of the polycaprolactone particles have a particle size that is less than 125 microns and wherein greater than 90 volume percent of the polycaprolactone particles have a sphericity that is greater than 0.75 and wherein the polycaprolactone particles have a moisture content that is adjusted to and maintained between 0.5 and 5% w/w.

13. A method of producing a polycaprolactone powder, the method comprising:
combining polycaprolactone and a polar organic solvent;
dissolving the polycaprolactone into the polar organic solvent to form a solution;
cooling the solution to a temperature that causes at least a portion of the dissolved polycaprolactone to precipitate from the solution;
separating precipitated polycaprolactone from the solution;
washing the separated, precipitated polycaprolactone to form a washed polycaprolactone; and
drying the washed polycaprolactone to form a dry polycaprolactone.

14. The method of claim 13, wherein the polar organic solvent is selected from the group consisting of: ethyl acetate, ethyl lactate, γ-valerolactone, N,N-dimethylformamide (DMF), N-methyl-2-pyrrolidone (NMP), tetrahydrofuran (THF), dichloromethane (DCM), chloroform; acetone, and dimethyl sulfoxide (DMSO).

15. The method of claim 13, wherein the polar organic solvent is ethyl lactate.

16. The method of claim 13, further comprising heating the combined polycaprolactone and polar organic solvent.

17. The method of claim 13, further comprising, after the drying step, a second separating step comprising separating dry polycaprolactone particles having a particle size less than 150 microns from larger dry polycaprolactone particles to form a sized polycaprolactone.

18. The method of claim 13, further comprising adding a nucleator in powder form to the solution for the purpose of inducing precipitate formation.

19. The method of claim 13, further comprising adding a secondary solvent to the solution for the purpose of inducing precipitation of the PCL, the second solvent being miscible with the polar organic solvent but does not support the dissolution of PCL.

20. The method of claim 13, wherein greater than 90 volume percent of the sized polycaprolactone particles have a sphericity that is greater than 0.75.

21. The method of claim 13, wherein greater than 80 volume percent of the sized polycaprolactone particles have a sphericity that is greater than 0.80.

22. The method of claim 20, wherein the polycaprolactone particles have a moisture content that is adjusted to and maintained between 0.5% w/w and 5% w/w.

23. A method of producing a polycaprolactone powder, the method comprising:
combining polycaprolactone and a polar organic solvent;
dissolving the polycaprolactone and at least one nucleator into the polar organic solvent to form a solution;
cooling the solution to a temperature that causes at least a portion of the dissolved polycaprolactone to precipitate from the solution;
separating precipitated polycaprolactone from the solution;
washing the separated, precipitated polycaprolactone to form a washed polycaprolactone; and
drying the washed polycaprolactone to form a dry polycaprolactone.

24. The method of claim 22, further comprising heating the solution.

25. The method of claim 22, wherein the polar organic solvent is selected from the group consisting of: ethyl acetate, ethyl lactate, γ-valerolactone, N,N-dimethylformamide (DMF), N-methyl-2-pyrrolidone (NMP), tetrahydrofuran (THF), dichloromethane (DCM), chloroform; acetone, and dimethyl sulfoxide (DMSO).

26. The method of claim 22, wherein the polar organic solvent is ethyl lactate.

27. A method of additive manufacturing, the method comprising selectively melting or sintering adjacent polycaprolactone particles, wherein greater than 95 number percent of the polycaprolactone particles have a particle size that is less than 125 microns and wherein greater than 90 volume percent of the polycaprolactone particles have a sphericity that is greater than 0.75 and wherein the polycaprolactone contains a detectable amount of ethyl lactate.

28. An article comprising polycaprolactone particles, wherein greater than 90 volume percent of the polycaprolactone particles have a particle size that is between 20 microns and 150 microns and wherein the polycaprolactone particles contain a detectable amount of a solvent comprising at least one of a biocompatible solvent; or a bioresorbable solvent.

29. A medical product comprising polycaprolactone particles, wherein greater than 90 volume percent of the polycaprolactone particles have a particle size that is between 20 microns and 150 microns and wherein the polycaprolactone particles contain a detectable amount of a solvent comprising at least one of a biocompatible solvent; or a bioresorbable solvent.
